(19) United States
(12) Patent Application Publication
(10) Pub. No.: US 2025/0152068 A1
(43) Pub. Date: May 15, 2025

(54) IMPLANT AND WEARABLE DEVICE IMPROVEMENTS

(71) Applicant: Wyss Center for Bio and Neuro Engineering, Genève (CH)

(72) Inventors: Tiago Bertolote, Genève (CH); Matthew Lapinski, Genève (CH)

(21) Appl. No.: 18/840,814

(22) PCT Filed: Mar. 8, 2023
(86) PCT No.: PCT/IB2023/052206

§ 371 Date: Aug. 22, 2024

Related U.S. Application Data
(60) Provisional application No. 63/269,023, filed on Mar. 8, 2022.

(51) Int. Cl. A61B 5/293

(52) U.S. Cl. CPC A61B 5/293

(57) ABSTRACT

An implant for a neural interface system includes a housing including a base and a flexible portion. The implant further includes at least two anchor attachment structures disposed at the base and arranged at an angle of greater than about 90° and less than about 180° with respect to one another as defined from an approximate center of the base. The anchor attachment structures can be connected by a curved connecting member disposed at a patient-facing surface of the base.

### RELATED APPLICATION

This application claims the benefit of U.S. Provisional Application No. 63/269,023, filed on Mar. 8, 2022. The entire teachings of the above application are incorporated herein by reference.

### BACKGROUND

Neural interface systems are used for the monitoring, treatment, or both monitoring and treatment of various neurological disorders. Today, monitoring and intervention in brain activity is generally accomplished by devices placed external to the scalp or inside the skull of a patient. The former approach is problematic because daily re-pasting of electrodes to the skin is generally required, which is uncomfortable and cumbersome for patients. The latter approach, which can enable neurostimulation and can yield high resolution stable EEG recordings, requires an extensive surgery for the patient (i.e., a craniotomy) and direct contact between the electrodes of the system and the brain, which comes with a small yet serious surgical risk. For these and other reasons, chronic intracranial device applications with brain recording capabilities remain a palliative option, when all other possibilities have been exhausted.

Intracranial recordings, particularly over long periods of time, are useful for a variety of applications, including, for example, epilepsy monitoring, neurorehabilitation, psychiatry, and sleep medicine. There is a need to provide for chronic monitoring of global brain activity with systems that may be placed in a patient by less-invasive means than electrode implants placed through the skull of a patient, and that are less cumbersome and able to be worn for extended periods of time as compared with scalp electrodes.

### SUMMARY

Neural interface system components are provided that include features providing for improved attachment to a patient and improved wearability for the patient. Such neural interface systems are adapted for subcutaneous and epiosteal placement in a patient.

An implant for a neural interface system includes a housing including a base and a flexible portion. The implant further includes at least two anchor attachment structures disposed at the base and arranged at an angle of greater than about 90° and less than about 180° with respect to one another as defined from an approximate center of the base.

The anchor attachment structures can be connected by a curved connecting member disposed at a patient-facing surface of the base.

Each of the two anchor attachment structures can include an attachment leg comprising an inflection point to deflect at least a portion of the anchor attachment structure toward a patient-facing surface of the base.

The housing is generally configured for subcutaneous placement against a skull of a patient, and the anchor attachment structures are configured to provide for anchoring of the housing to the skull.

The implant can further include electronics c configured to record electroencephalography (EEG) signals detected from sensors of the leads (e.g., 32 channels of electrical brain activity detected by the leads can be recorded). The electronics can be further configured to transmit the recorded electroencephalography (EEG) signals to an extracutaneous wearable device (alternatively referred to as a wearable headpiece). The recording and transmitting of the EEG signals can be continuous.

A lead connection area can be included at the body of the implant and can be configured to engage leads extending from the implant. For example, the lead connection area can include at least four lead connection structures arranged in a substantially flat configuration. The implant can further include a magnetic alignment component configured to align an extracutaneous wearable device to the implant. The magnetic alignment component can be removeable.

A wearable device of a neural interface system includes a housing and a removeable cover configured to enclose at least a patient-facing surface of the housing.

The removeable cover can include a plurality of indentations, a plurality of protrusions, or a combination thereof. For example, the indentations and/or protrusions can be paths extending from an interior location of a patient-facing surface of the cover toward a perimeter of the patient-facing surface of the cover. The paths can extend radially from a center of the patient-facing surface of the cover. The indentions and/or protrusions can be of a same size or of varying sizes. For example, the indentations and/or protrusions can include a plurality of differently-sized indentations and protrusions. The plurality of differently-sized indentations and/or protrusions can be arranged to increase in size from an interior location of a patient-facing surface of the cover toward a perimeter of the patient-facing surface of the cover, or vice versa.

The angle at which the two anchor attachment structures are disposed can be within range of about 130° to about 150°, or can be of about 145°. The anchor attachment structures can each be disposed on an anchor attachment leg that includes an inflection point, for example, an inflection point that orients each leg to deflect toward a patient-facing surface of the base. The anchor attachment legs can be deflected toward the patient-facing surface of the base by about 5° to about 30°, or by about 20°. The anchor attachment legs can each be flexible at their respective inflection points. Alternatively, or in addition, the anchor attachment legs can comprise a complaint material configured to conform to a curvature of a skull. The anchor attachment legs can comprise a material that resists or inhibits osseointegration. For example, the anchor attachment legs can each be formed from or include an overmold of such material, for example, polyetheretherketone (PEEK), polysulfone (PSU), polyethylenimine (PEI), thermoplastic polyurethane (TPU), or a combination thereof, which can assist in preventing osseointegration of the legs with patient tissue and can advantageously provide for easy explanting of the device.

The flexible portion of the implant can include an inflection point. The inflection point can orient the flexible portion to deflect toward a patient-facing surface of the base, in other words, toward a skull of a patient wearing the implant. For example, the flexible portion can be deflected toward the patient-facing surface of the base by 1° to about 90°, or by about 5° to about 45°, or by about 15°. The flexible portion can include multiple inflections and/or a curved geometry.

A method of implanting a component of a neural interface system includes creating an incision in a scalp of a patient, the incision being of about 30 mm to about 50 mm in length, and, through the incision, placing an implant comprising a housing including a base and a flexible portion subcutaneously in a subscalp space against the skull of the patient. The incision can be, for example, a curved incision. By not requiring a recess to be formed in the skull to insert the system into, the incision can be smaller. The system does not need to penetrate the skull since the system can be thin enough to fit under the scalp.

The method can further include securing the implant to the skull of the patient at at least one attachment structure disposed at the base of the implant. For example, the at least one attachment structure can include two attachment structures arranged at an angle of greater than 90° and less than 180° with respect to one another.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic of a neural interface system.

FIG. 2A is a schematic of an example implant of a neural interface system.

FIG. 2B is a side view of the implant of FIG. 2A.

FIG. 2C is a bottom view of the implant of FIG. 2A.

FIG. 2D is a view of a patient-facing surface of the implant of FIG. 2A.

FIG. 3 illustrates an example positioning of an implant with respect to a patient.

FIG. 4 is a schematic of an example wearable of a neural interface system.

FIG. 5A is a schematic of an example cover for a wearable.

FIG. 5B is another view of the example cover of FIG. 5A.

FIGS. 5C-5J are front, back, top, bottom, right side, left side, top perspective and bottom perspective views of another example cover.

FIG. 6A is a schematic of another example cover for a wearable.

FIG. 6B is another view of the example cover of FIG. 6A.

FIGS. 6C-6J are front, back, top, bottom, right side, left side, top perspective and bottom perspective views of another example cover.

FIG. 7 is a schematic of an example implant with a lead connection structure providing for a substantially flat lead configuration.

FIG. 8 is a schematic of an example implant with attachments legs having multiple inflections.

FIG. 9 is a schematic of an example implant with a lead connection structure providing for a bundled lead configuration.

FIG. 10 is a schematic of an example implant with a lead connection structure providing for a stacked lead configuration.

FIG. 11A is a side view of the implant shown in FIG. 7 and having a flexible portion with a single inflection.

FIG. 11B is another side view of the implant shown in FIGS. 7 and 11A illustrating relative curvature of the flexible portion.

FIG. 12A is a side view of another example implant, the implant having a flexible portion with two inflections.

FIG. 12B is side view of the implant shown in FIG. 12A, further illustrating relative curvature of the flexible portion.

FIG. 13A is a schematic of an example cover engage with a wearable device of a neural interface system.

FIG. 13B is a schematic of the cover of FIG. 13A without engagement with a wearable device.

FIG. 14 is a schematic of an example patient-facing surface of a cover for a wearable device.

FIGS. 14A-14H are front, back, top, bottom, right side, left side, top perspective and bottom perspective views of another example cover.

FIG. 15 is a schematic of another example of a patient-facing surface of a cover for a wearable device.

FIGS. 15A-15H are front, back, top, bottom, right side, left side, top perspective and bottom perspective views of another example cover.

### DETAILED DESCRIPTION

A description of example embodiments follows.

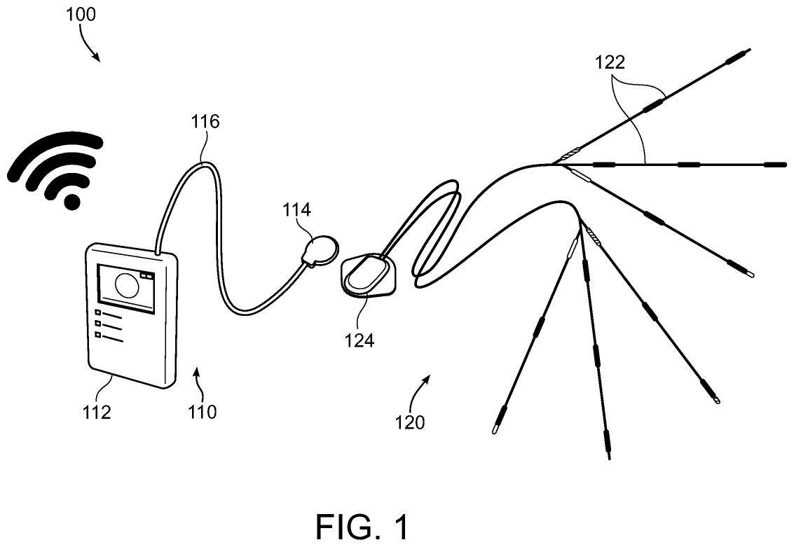

An example of a neural interface system is shown in FIG. 1. The neural interface system 100 includes a component 110 that is worn externally of a patient and a component 120 that is internally implanted in the patient. The external component 110 includes a wearable device 114 connected to a recorder 112 by a cable 116. The implantable component 120 includes leads 122 connected to an implant 124. The leads can be in the form of one or more tridents for placement about a skull of a patient. The implantable component can be configured for epiosteal and subcutaneous placement in a patient.

The neural interface system 100 can be used to continuously record subcutaneous electroencephalography (EEG) signals from a patient. Briefly, the leads 122 include EEG sensors that are configured for epiosteal and subcutaneous placement in a patient. The implant 124 can be an implantable telemetry unit (ITU), which can capture EEG signals detected by the leads 122 and transmit the captured EEG signals through the skin of the patient to the wearable device 114. The wearable device 114 can be an electronic device worn on the body of the patient. The wearable device 114 can power the implant 124 and receive EEG data from the implant. The received EEG data can then be stored on the recorder 112, where it may be further transmitted (e.g., wirelessly) to other devices.

Further description of an example neural interface system can be found in WO 2019/211314, entitled “Neural Interface System,” the contents of which are incorporated by reference herein in their entirety.

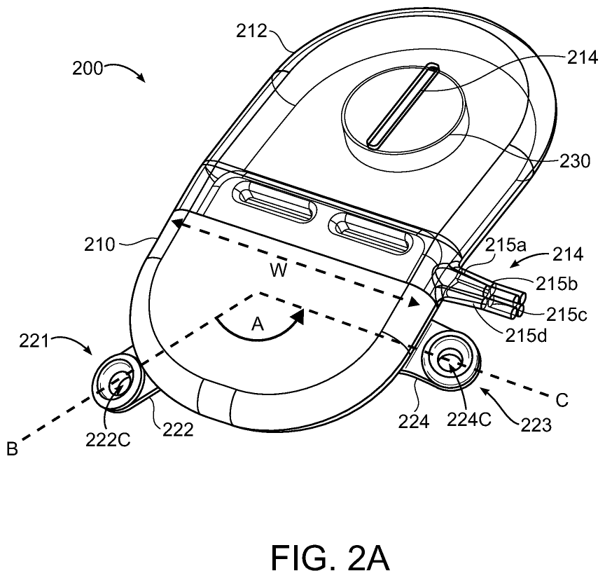

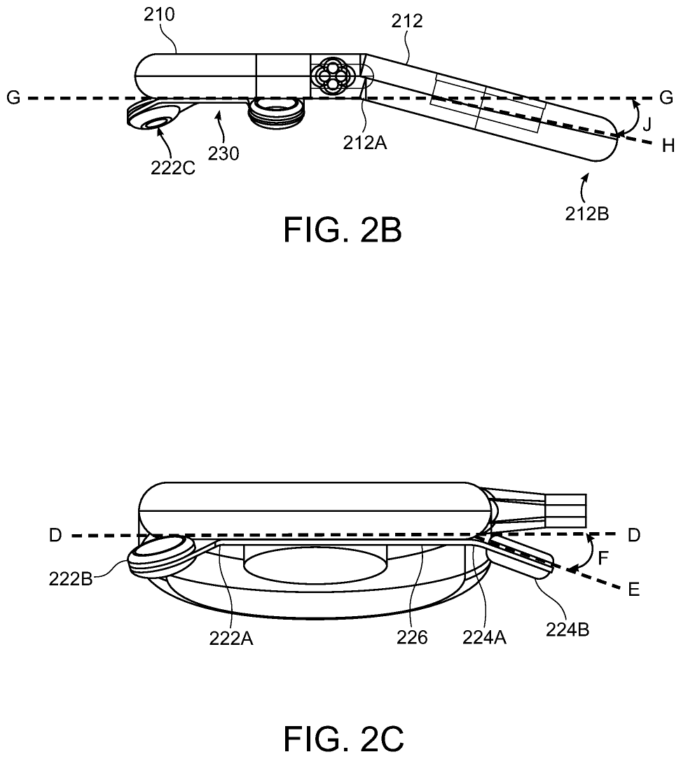

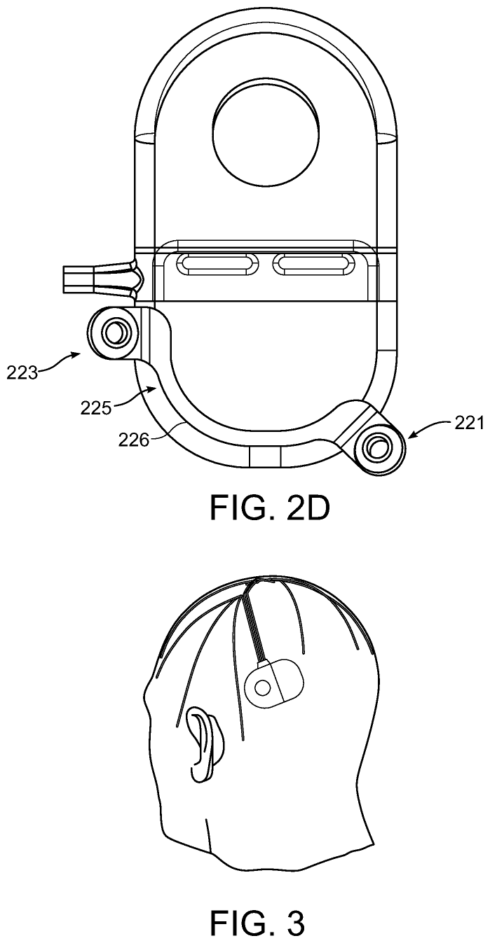

An example of an implant of a neural interface system is shown in FIG. 2A. The implant 200 includes a base 210 and a flexible portion 212, which can be, for example, a flexible sleeve or overmold. The base 210 can house electronic components for registering, digitizing, and/or transmitting EEG data. The base can be formed of a rigid biocompatible material to provide for secure encapsulation of the electronics, such as a ceramic, epoxy, metal, rigid polymer, or combination thereof. The base may be overmolded, such as with an elastomeric material, with the elastomeric material then forming a continuous body that also forms the flexible portion of the implant 200. Examples of suitable elastomeric materials include silicone, polyurethane rubber (PUR), and thermoplastic elastomers (TPE). Alternatively, the flexible portion may be attached to the base. The leads of the system (e.g., leads 122) can be connected to the electronics housed in the base 210 of the implant at a lead connection area 214. The lead connection area 214 can include lead connection structures 215a-215d arranged to maintain leads in a compact configuration. As illustrated, the lead connection area 214 comprises four lead connection structures 215a-215d that are bundled. Alternatively, the lead connection structure can provide for a side-by-side arrangement of the leads with respect to one another.

The lead connection area 214 can provide for connection of a plurality of leads, for example, up to 4 cables, in a compact configuration. The leads (e.g., leads 122) can comprise sensors configured to provide, for example, 32 channels of electrical brain activity.

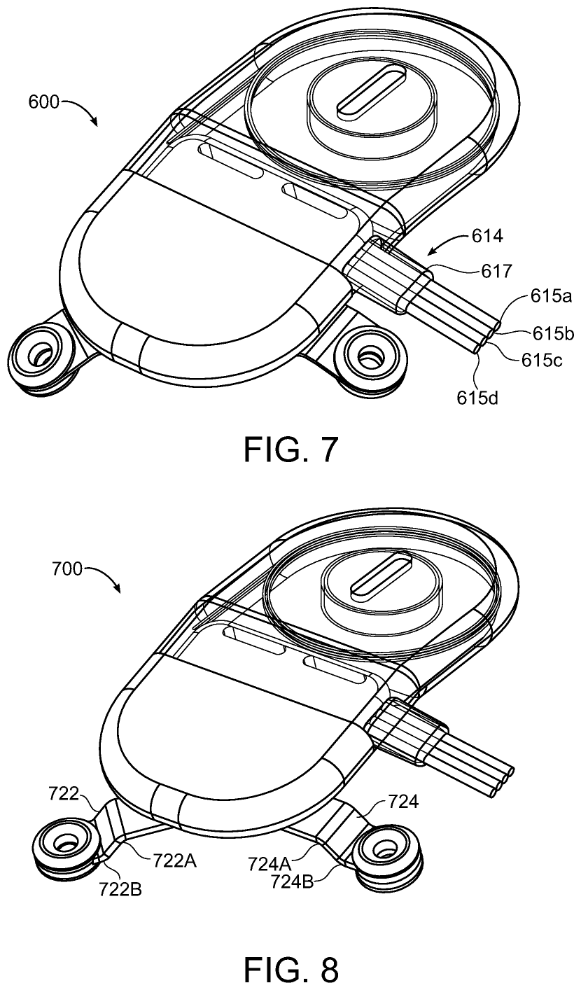

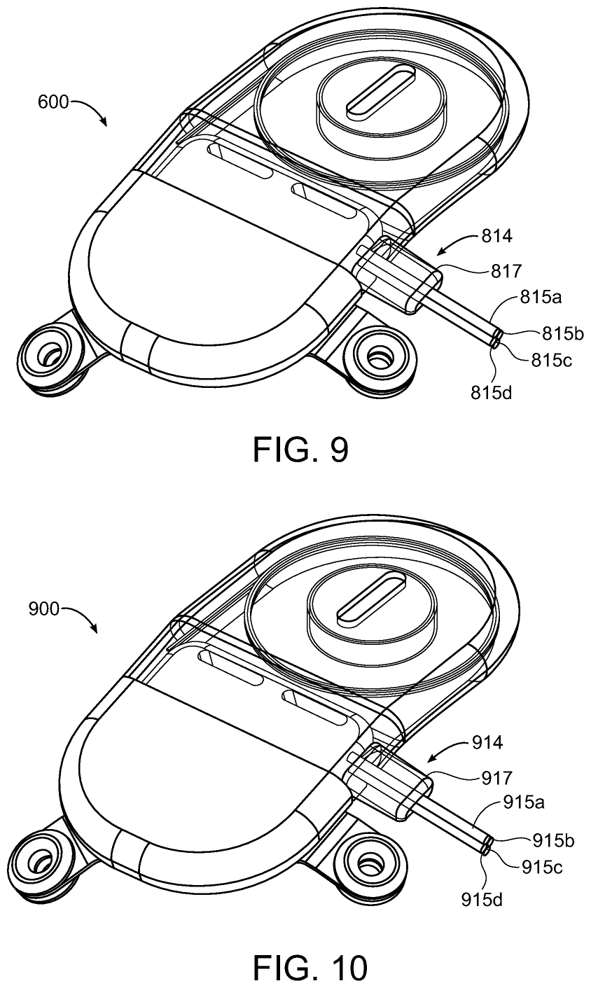

Additional examples of structural configurations for a lead connection area of an implant are shown in FIGS. 7, 9, and 10. As illustrated with the implant 600 in FIG. 7, a lead connection structure 614 provides for connection of leads 615a-d in a substantially flat configuration. A substantially flat configuration can assist in maintaining a minimal overall height of the implant. A lead connection structure 814 providing for connection of leads 815a-d in a bundled configuration is shown with the implant 800 of FIG. 9, and a lead connection structure 914 providing for connection of leads 915a-d in a stacked configuration (e.g., a two-by-two configuration for four leads) is shown with the implant 900 of FIG. 10. The bundled and stacked configurations can assistant in maintaining a minimal width of the lead connection area. In contrast with the lead connection structures 215a-215d shown in FIG. 2A, the lead connection structures 614, 814, 914 of FIGS. 7, 9, and 10 provide for a projecting portion 617, 817, 917 that is relatively smooth, providing for easier manufacture while still minimizing a footprint of a lead connection area. Lead connection structures can be formed as part of the flexible sleeve (e.g. flexible portion 212 in FIG. 2A) of the device.

The base of the housing can include at least two anchor attachment structures 221, 223. The anchor attachment structures can be provided by an integral structure, such as an anchoring element 225 (FIG. 2D) that includes anchor attachment legs 222, 225 with a connecting member 226 therebetween, or can include distinct anchor attachment legs extending from the base. The anchor attachment legs 222, 224 are included at the base housing 210 for securing the implant to a patient. The anchor attachment legs 222, 224 can be arranged at an angle with respect to one another to provide for two distinct attachment locations for securing the implant to the patient. For example, at least two anchor attachment legs can be arranged at an angle of greater than about 90° and less than about 180° with respect to one another as defined from an approximate center of the base 210.

As illustrated in FIG. 2A, the anchor attachment legs 222, 224 are disposed at an angle A of about 1450 with respect to one another. In particular, one anchor attachment leg 224 is disposed along axis C, which is substantially parallel to a horizontal width W of the implant, and another anchor attachment leg 222 is disposed along axis B. While more than two anchor attachment legs may be included at the base 210 of an implant, at least two anchor attachment legs can be included which are disposed at an obtuse angle with respect to one another. In particular, the anchor attachment legs 222, 224 can be arranged at angle of greater than 90° and less than 180° with respect to one another. For example, the angle A can be within range of about 120° to about 160°, or of about 130° to about 150°, or of about 135° to about 145°. Such an arrangement can advantageously provide for secure attachment of the base 210 of the implant to a patient while minimizing an incision size required to place and secure the implant to the patient. For example, with such an arrangement, an incision size of about 30 mm to about 50 mm in length, or of about 45 mm in length, can be sufficient for providing access for implanting the device, thereby minimizing surgical invasiveness for the patient. At least one of the anchor attachment legs can be disposed at or near a lead connection area 214, as shown in FIG. 2A, to prevent or minimize the effect of movement of the implant on the leads, or vice versa.

For context, sub-scalp implants are typically inserted through an incision made in the patient's scalp. While pressure between the scalp and the skull can provide for some retention and position control over an implant once it is inserted into a patient, it is desirable to minimize or eliminate movement of the implant, which can create patient discomfort and can affect positioning of the leads to which the implant is attached. As such, one or more anchor attachment structures can be included on the implant to secure the implant to the patient's skull. A single anchor attachment structure can be insufficient because a single point of attachment may still permit for rotational movement of the implant about the single anchor point, as well as other movement at other ends of the device. However, to anchor the implant at opposing sides of the device requires a significantly larger incision size as the incision must then not only accommodate the width of the implant, but also adequate space to fit and secure the implant on either side of the device. The inclusion of anchor attachment structures in or on the implant, as illustrated in FIGS. 2A-2C, can advantageously minimize an incision size while providing for two anchoring locations that can securely retain the implant in position.

As further illustrated in FIGS. 2B and 2C, the anchor attachment legs 222, 224 can each include an inflection point 222A, 224A. The inflection points 222A, 224A orient the anchor attachment legs 222, 224 to deflect from a plane D defined by a patient-facing surface 230 of the base 210. For example, the inflection point 224A orients at least a portion of the anchor attachment leg 224 along an axis E, which is at an angle F of about 5° to about 40°, or of about 10° to about 25°, with respect to plane D and deflecting the leg toward a patient-facing surface of the device (i.e., toward a skull when the device is worn by a patient). The anchor attachment legs 222, 224 can be flexible at their respective inflection points to provide for an adaptable and secure fit to a patient's skull. While each leg is illustrated as having a single inflection point, more than one inflection point can be included, or the leg can include a curved geometry. For example, the implant 700 shown in FIG. 8 includes attachment legs 722, 724, each of which includes two inflection points 722A-B and 724A-B.

As illustrated in FIGS. 2B-2D, the anchor attachment legs 222, 224 can be connected by a connecting member 226; however, the anchor attachment legs may be independently secured to the base 210 without a connecting member 226. The anchor attachment legs 222, 224 and the connecting member 226 can be unitarily or distinctly formed by a metal material (e.g., titanium) which can be integral with or welded to the base 210 of the device while also providing for a degree of flexibility for bending at the inflection points 222A, 224A during implantation of the device. Alternatively, the anchor attachment legs can be formed by overmold material, such as by an overmold that provides for the flexible portion 212 of the device and can optionally encapsulate the base 210 of the device. The angle at which the anchor attachment legs 222, 224 are oriented can accommodate curvature of a skull. For example, the angle F at which the anchor attachment legs are deflected can be within range of about 5° to about 25°, or of about 10° to about 20°.

The connecting member 226 can be a curved member, as illustrated in FIG. 2D. A curved connecting member 226 can provide for an anchoring element 225 that can be easily manufactured and that can provide for support and secure connection of the anchor attachment structures 221, 223 to the implant with a minimal amount of fabrication material (e.g., titanium). As illustrated, the connecting member 226 is curved such that a general shape of the connecting member follows a curved perimeter or edge of the patient-facing surface of the base 210. The curved connecting member 226 can provide for a generally flush attachment of the anchoring element 225 to the patient-facing surface of the base 210. As best shown in FIG. 2C, the inflection points 222A and 224A can be disposed at an edge of the base 210, which can assist in minimizing an incision size for implanting the device by maintaining the anchor attachment structures 221, 223 close to the base while still permitting the attachment structures to flex.

The anchor attachment legs 222, 224 can be formed from or include a material that resists (e.g., inhibits, prevents, delays) osseointegration. For example, as illustrated, the anchor attachment legs 222, 224 include overmolds 222B, 224B. The overmolds can comprise a material that prevents osseointegration of the anchor attachment legs 222, 224 with patient tissues, including the skull. The overmolds can be, for example, polyetheretherketone (PEEK), polysulfone (PSU), polyethylenimine (PEI), thermoplastic polyurethane (TPU), or a combination thereof. As illustrated in FIGS. 2A-2C, the overmolds are disposed through screw cavities 222C, 224C and extend over upper and lower surfaces of the anchor attachment legs 222, 224. Alternatively, or additionally, the overmolds can be disposed about a perimeter of the attachment legs 222, 224. The overmolds can completely or partially cover upper and lower surfaces of the anchor attachment legs 222, 224.

The flexible portion 212, alternatively referred to as a sleeve, can house coils (not shown in FIG. 2) for wireless power transmission when the implant is in close proximity to a wearable (e.g., wearable 114). The sleeve can be formed of a material that permits for infrared (IR), radiofrequency (RF), or other transmission of EEG signals to the wearable, such as silicone, polyurethane rubber (PUR), and thermoplastic elastomers (TPE). The sleeve can optionally include an opening 214 through which a magnet 230 can be inserted and removed. The magnet 230 can provide for removeable attachment of a wearable device (e.g., wearable device 114, alternatively referred to as a headpiece) at the location at which the implant is disposed in the patient. The sleeve 212 can be formed of a compliant or flexible material. For example, the material can be of about 10 to about 80 on the Shore 00 scale. The compliant material can be configured to conform to a curvature of a skull. A flexible sleeve can provide for comfort, ease of positioning in a patient, and magnet accessibility through opening 214.

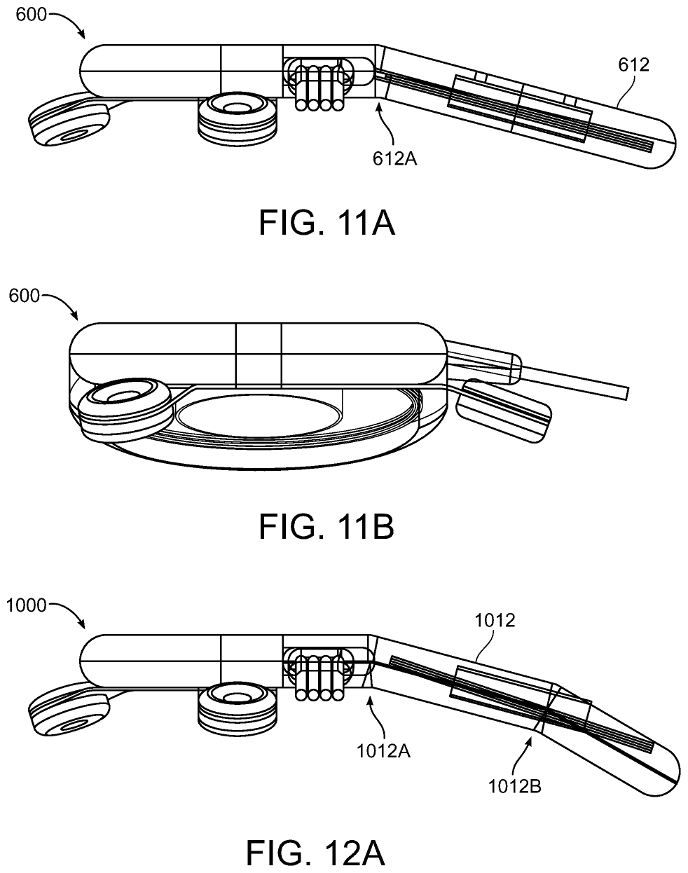

As best illustrated in FIG. 2B, the sleeve 212 can include an inflection point 212A. The inflection point 212A orients at least a portion of the sleeve to deflect from a plane G defined by a patient-facing surface 230 of the base 210. For example, the inflection point 212A orients at least a portion of the sleeve along an axis H, which is at an angle J of about 15° with respect to plane G. The sleeve 212 can be flexible at one or more inflection points, for example, at the inflection point 212A, to provide for an adaptable and secure fit to a patient's skull. The angle at which the sleeve 212 is oriented can accommodate curvature of a skull. For example, the angle J at which the sleeve is deflected can be within range of about 1° to about 90°, of about 5° to about 25°, or of about 10° to about 20°. Alternatively, or in addition, the sleeve can include multiple inflection points or a curved geometry, including, for example, complex curves or multiple inflection points at one or more locations along a length of the sleeve. Examples of structural configurations for an inflected sleeve of an implant are shown in FIGS. 11A-12B. The implant 600 of FIGS. 11A-B includes a sleeve 612 having one inflection point 612A. The implant 1000 of FIGS. 12A-B includes a sleeve 1012 having two inflection points 1012A, B. An example placement of an implant 200 at the skull of a subject, including associated leads, is shown in FIG. 3.

The deflection of the flexible sleeve in combination with the anchor attachment legs can provide for at least three points of connection for retaining the implant securely in place. For example, pressure exerted by the flexible sleeve at an end 212B of the device due to the deflection of the sleeve can provide for frictional engagement with the skull. The deflection of at least a portion of the sleeve 212 of the implant can thereby assist in securing the implant to the patient at an opposing end of the device from which the anchor attachment legs are located. With the at least two anchor attachment legs 222, 224 disposed at an angle (e.g., an obtuse angle) with respect to one another at the base 210 of the device and with the deflection of the flexible sleeve 212 at the end 212B of the device, a substantially triangular arrangement of attachment points can be provided. Such a triangular arrangement can provide for secure attachment to a subject while minimizing an incision size and minimizing a number of screw attachment locations to a patient.

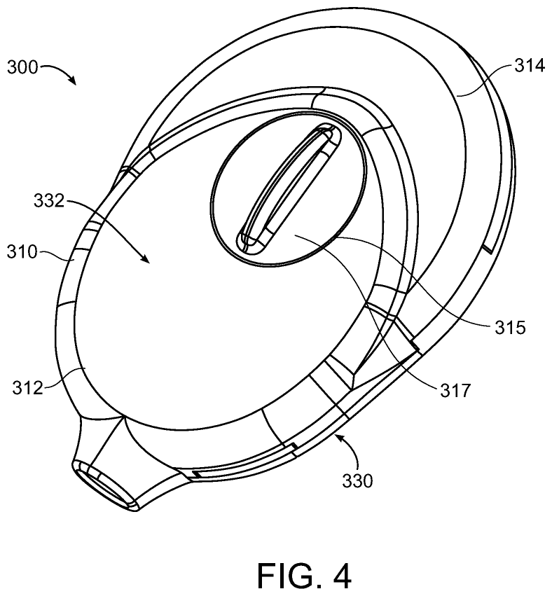

An example of a wearable device configured to engage with an implant, such as implant 200, is shown in FIG. 4. The wearable device 300 can include a housing 310. Optionally, the housing 310 can include an opening 315 that provides access to a magnet (not shown) contained therein. As illustrated, the opening is configured to receive a removeable component 317, such as a screw-in disc, a slideable cover, or a hinged cover, to securely enclose the magnet within the housing while providing access to the magnet for insertion and removal. The housing 310 can be of a complimentary size and shape for engaging with an implant. As illustrated, a portion 312 of the housing 310 at which electronics of the device are housed is configured to overlap, at least in part, with base 210 of the implant 200. Another portion 314 of the housing can house coils (not shown) for wireless power transmission to the implant. The portion 314 of the housing can be reduced in size with respect to the portion 312 to minimize an overall size of the wearable.

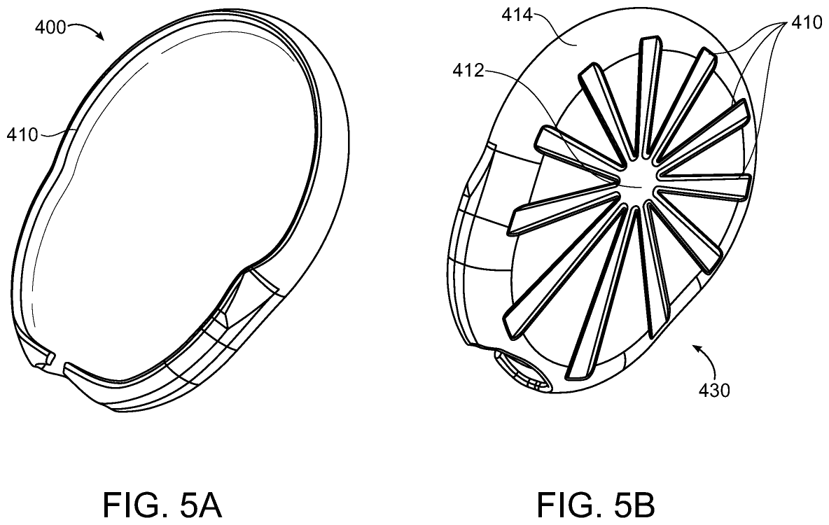

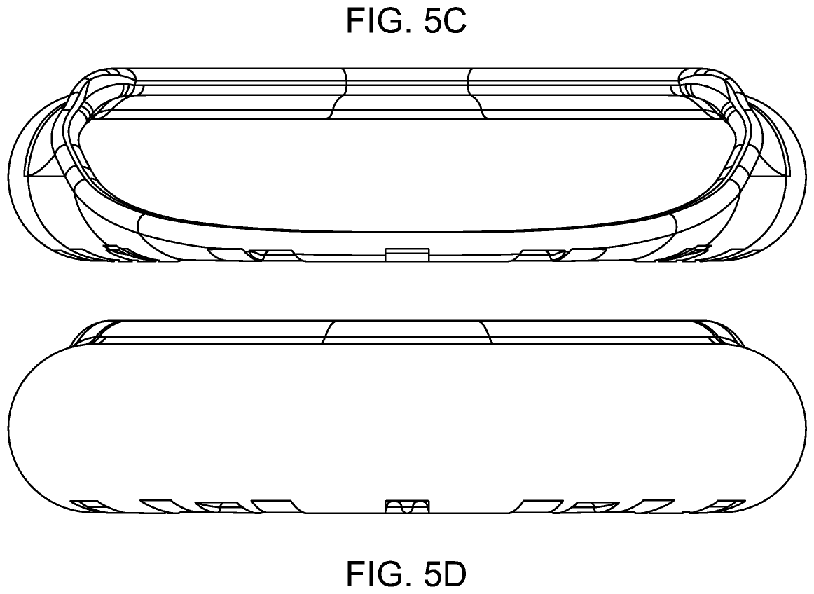

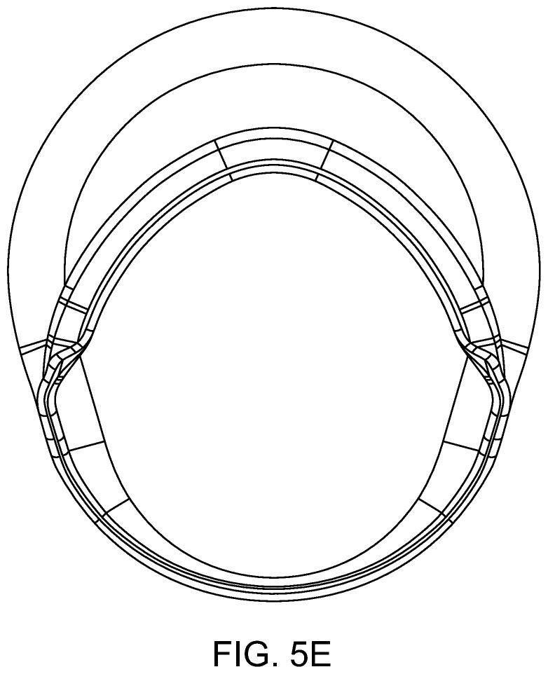

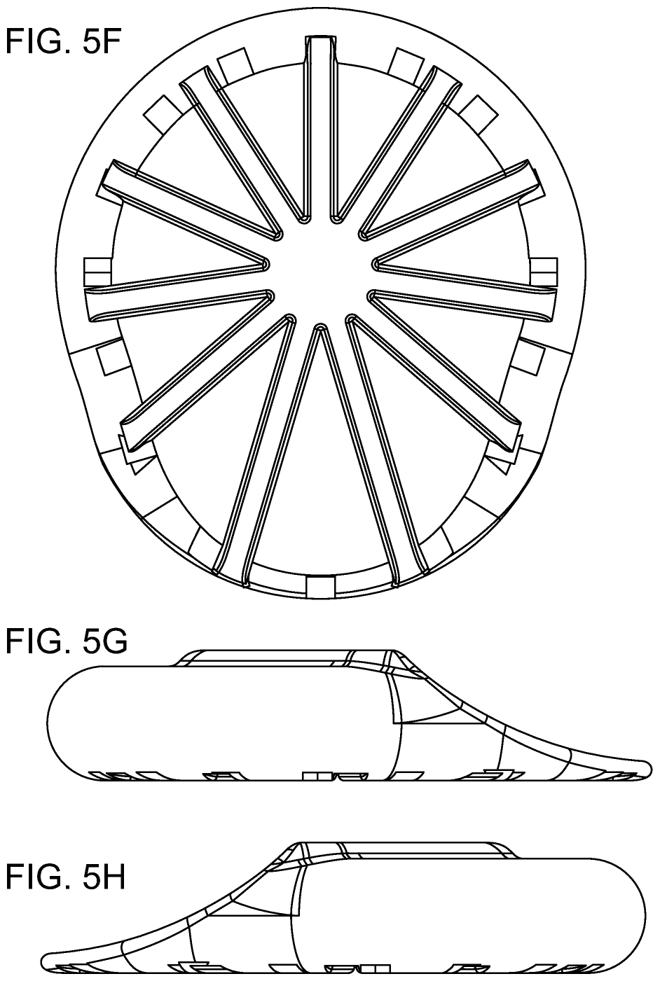

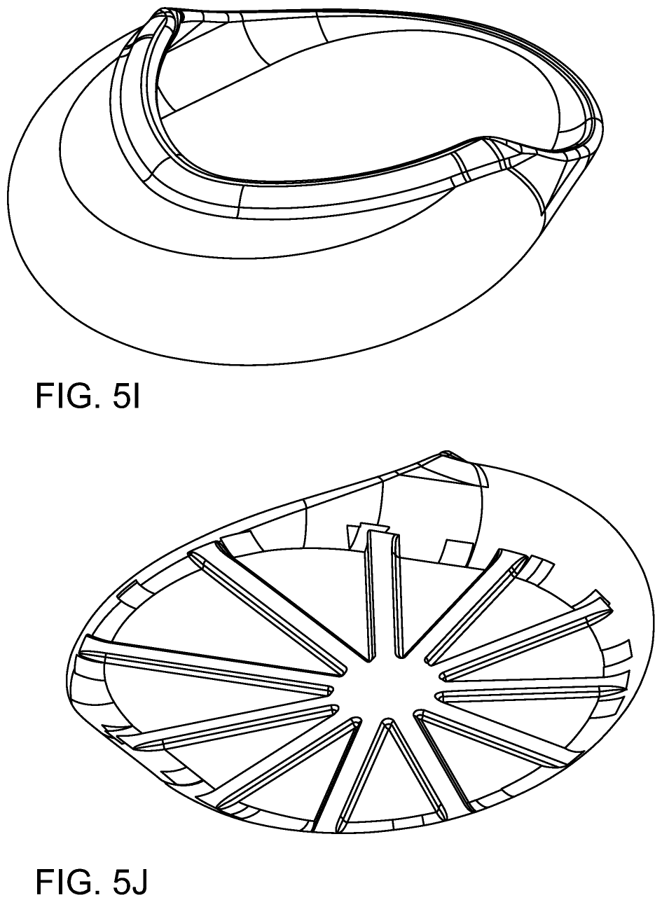

The wearable can further include a removeable cover. An example of a removeable cover is shown in FIGS. 5A-5B. The removeable cover 400 can be configured to enclose at least a patient-facing surface 330 of the wearable (FIG. 4). For example, as illustrated in FIG. 5A, the removeable cover 400 is configured to cover a patient-facing surface 330 of the wearable 300 while leaving a non-patient-facing surface 332 of the wearable substantially exposed. The removeable cover 400 includes a lip 410 configured to engage with a perimeter of the wearable 300 to provide for its attachment and removal to the wearable. Another example of a removable cover is shown in FIGS. 5C-5J.

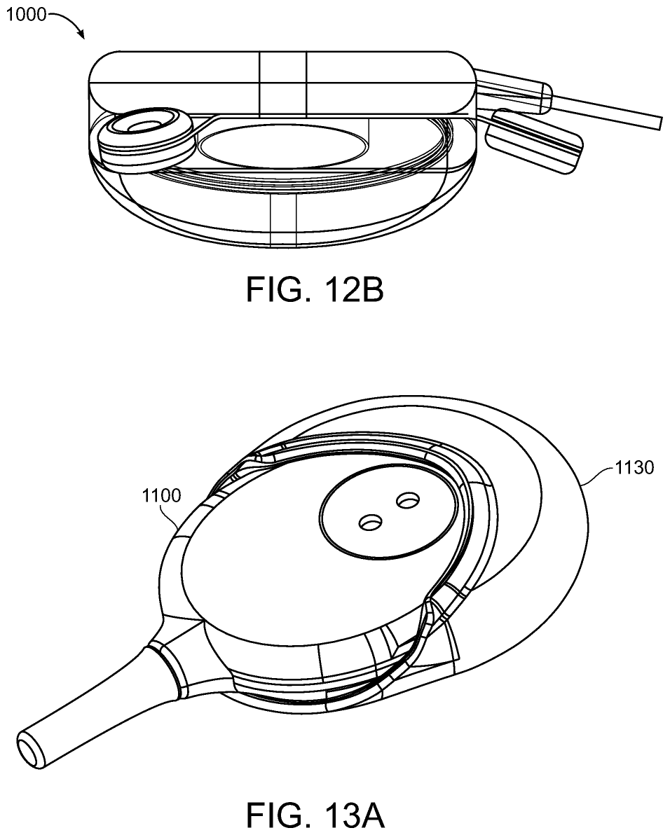

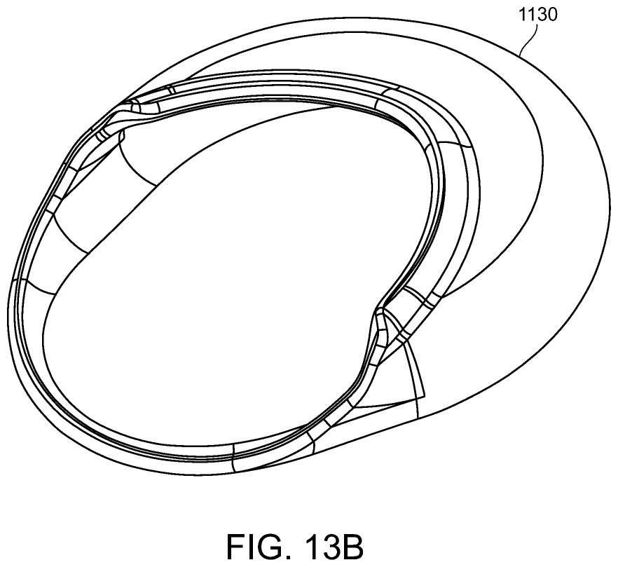

An additional example of a wearable cover engaged with a wearable device is shown in FIGS. 13A-B. The cover 1130 is configured to encase an upper portion of the wearable device 1100 and provide a patient-facing surface 1113.

As illustrated in FIG. 5B, a patient-facing surface 430 of the cover includes a plurality of indentations 410. The indentations 410 can be in the form of paths extending radially from an interior location 412 of the patient-facing surface 430 toward a perimeter 414 of the surface 430.

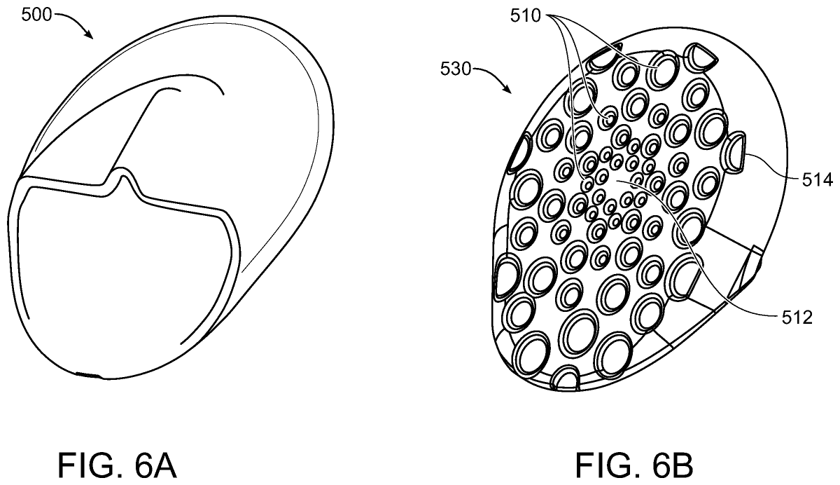

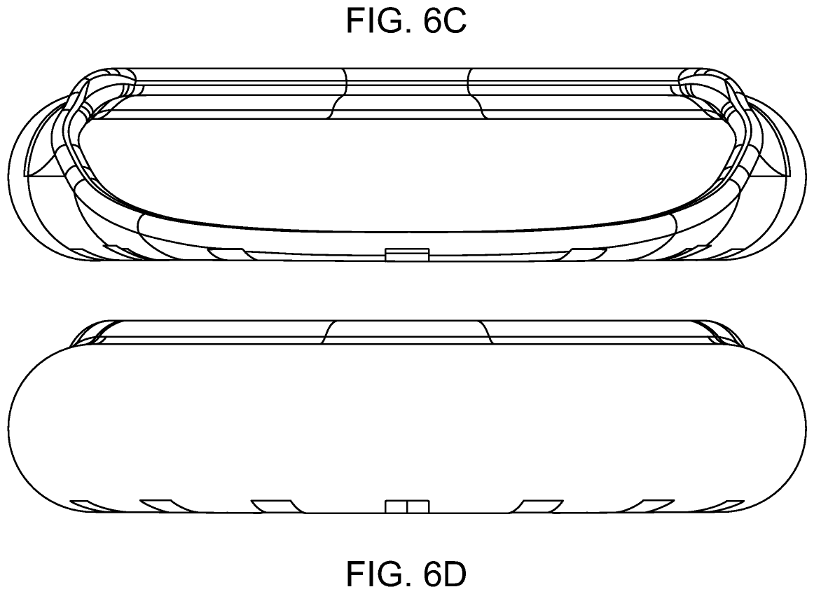

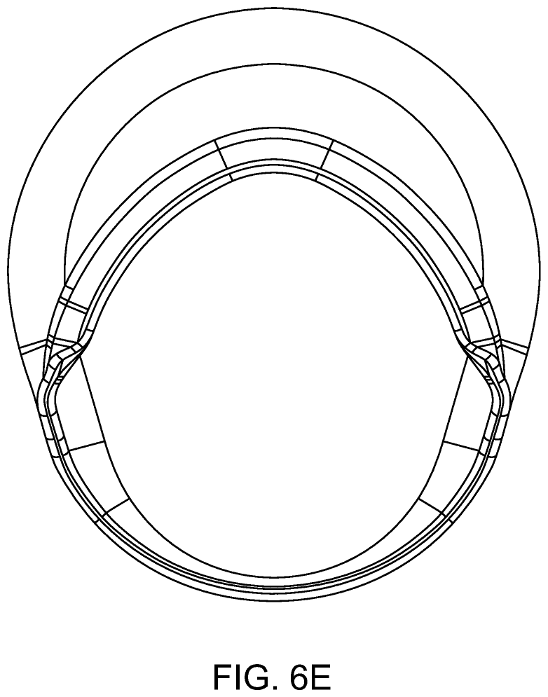

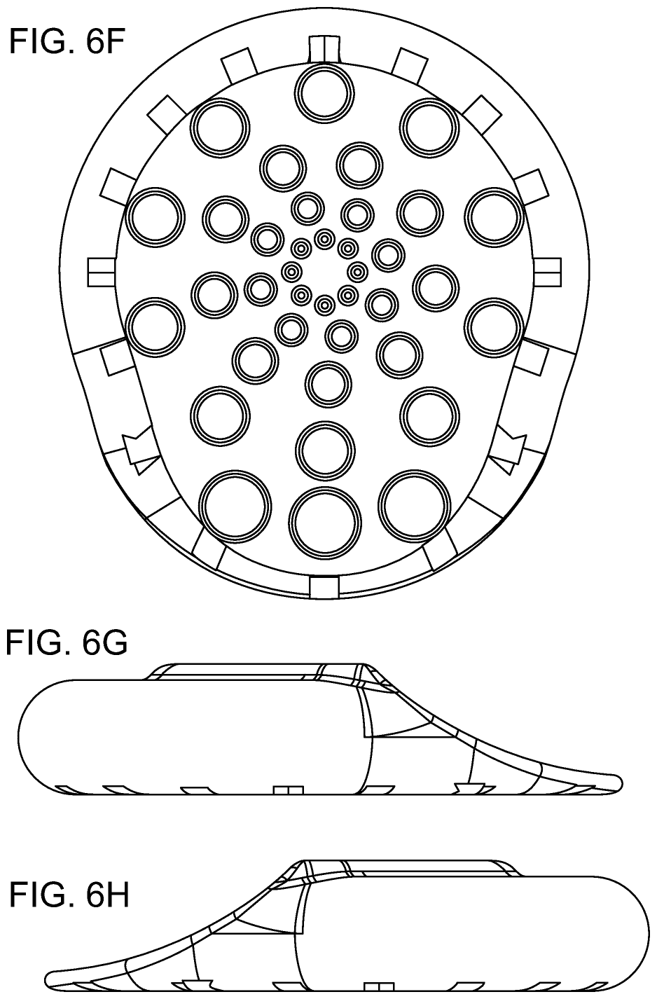

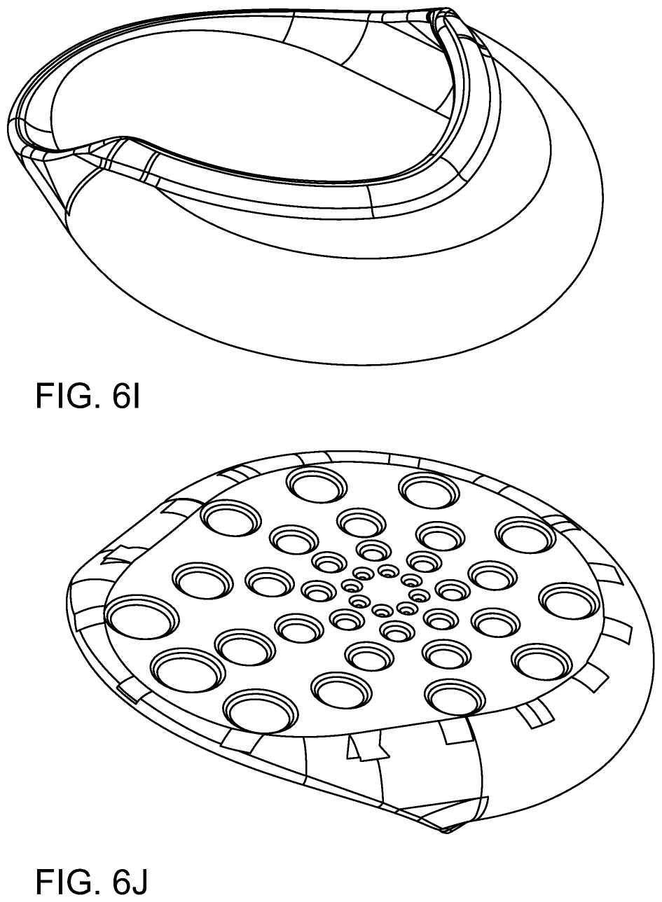

Another example of a removeable cover is shown in FIGS. 6A and 6B. The removeable cover 500 encloses both a patient-facing surface 330 and at least a portion of a non-patient-facing surface 332 of a wearable 300. As illustrated, the removeable cover 500 includes a plurality of indentations 510 of varying sizes. The indentations can be arranged in a radial pattern about an interior location 512 of the patient-facing surface 530 of the cover. For example, the indentations 510 can be arranged to increase in size from the interior location 512 of the surface 530 toward a perimeter 514 of the surface 530, or vice versa. Another example of a removable cover is shown in FIGS. 6C-6J.

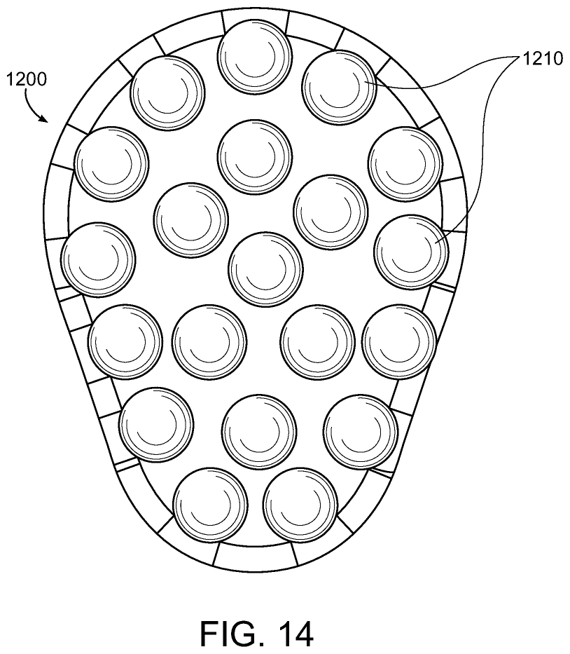

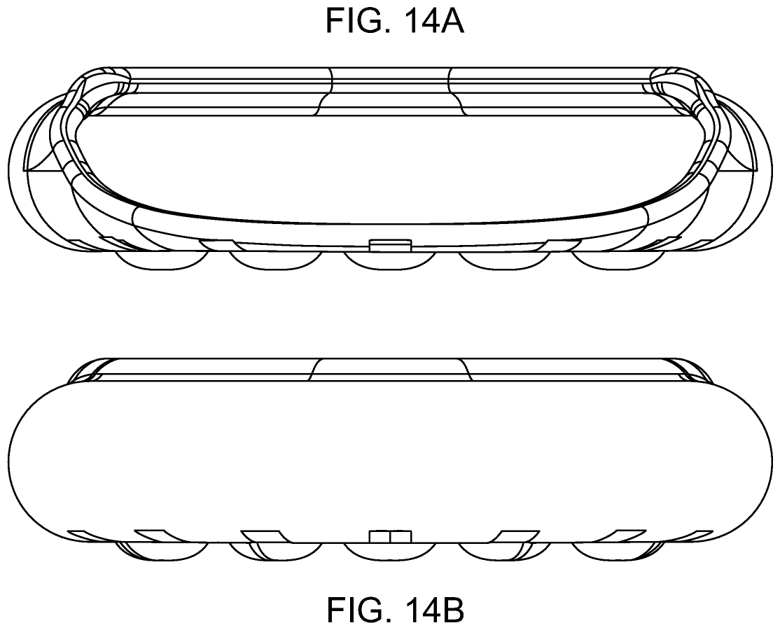

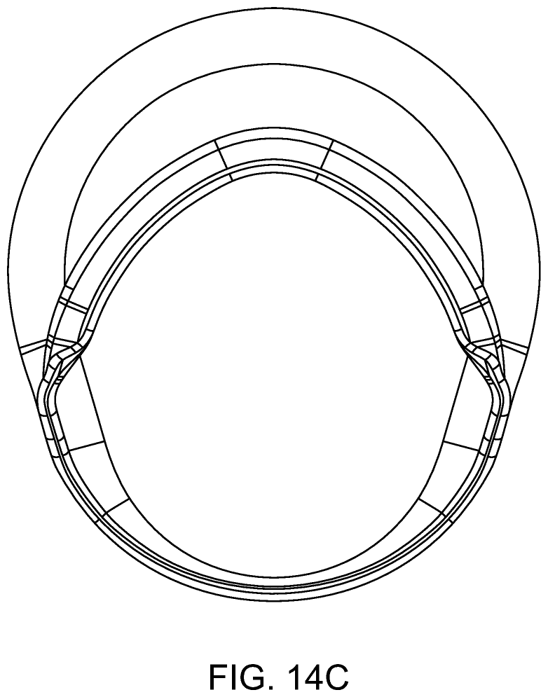

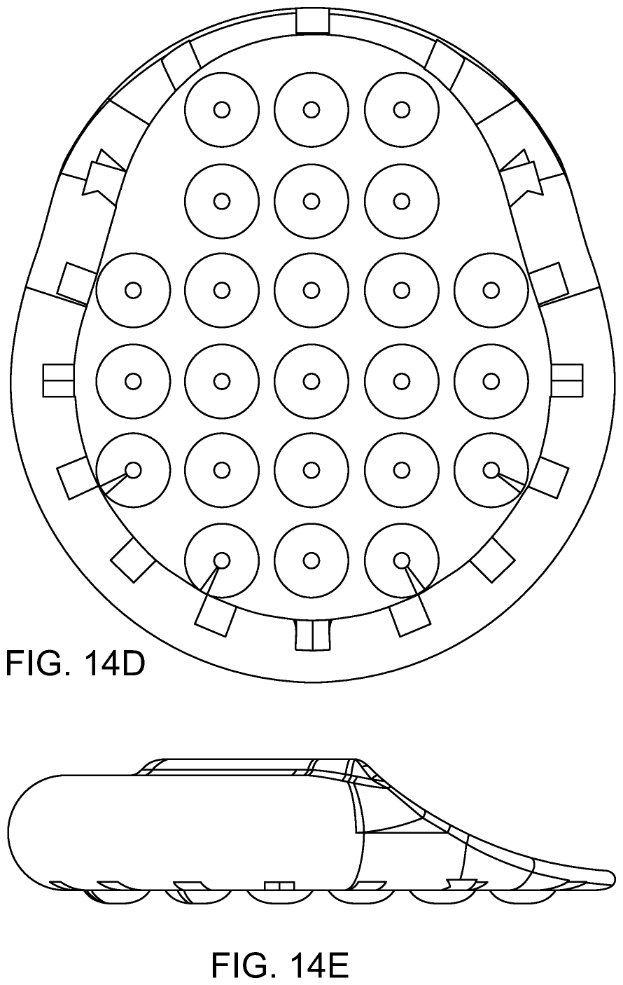

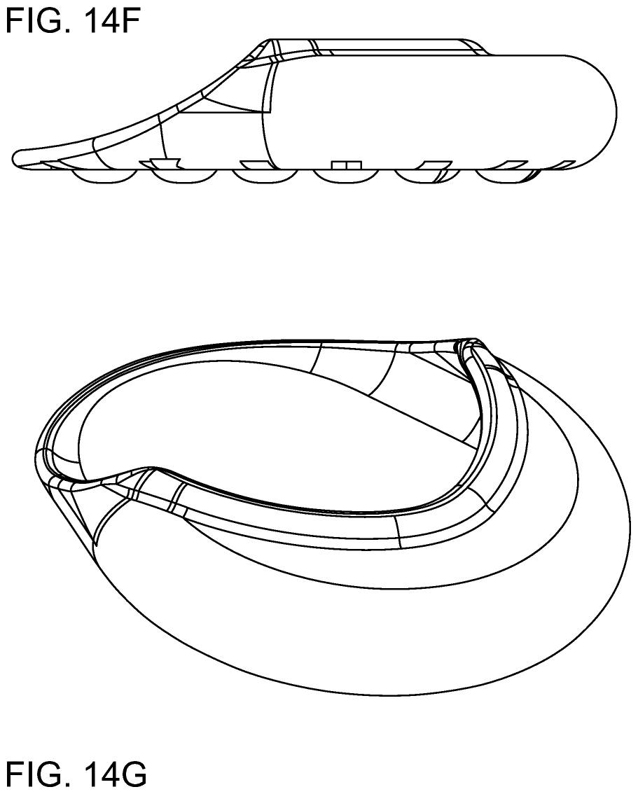

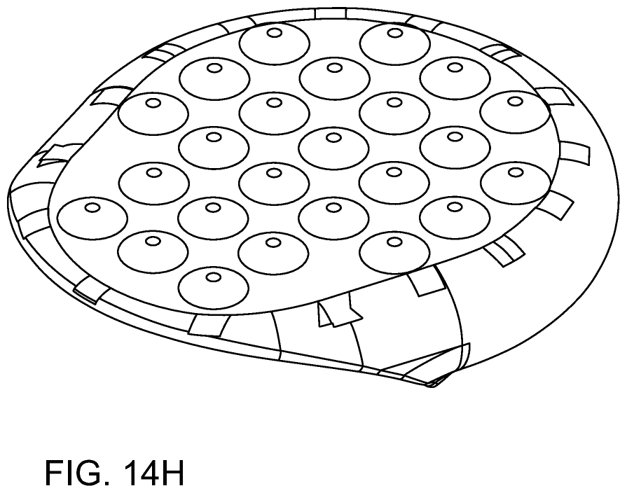

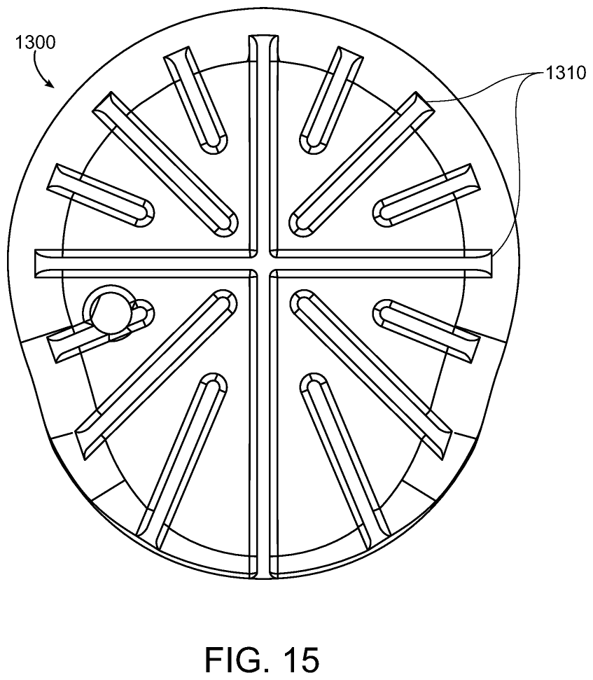

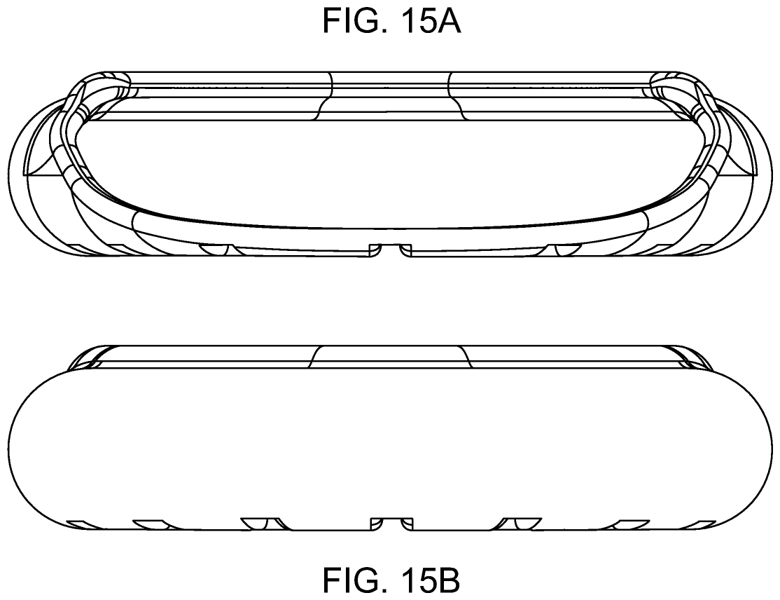

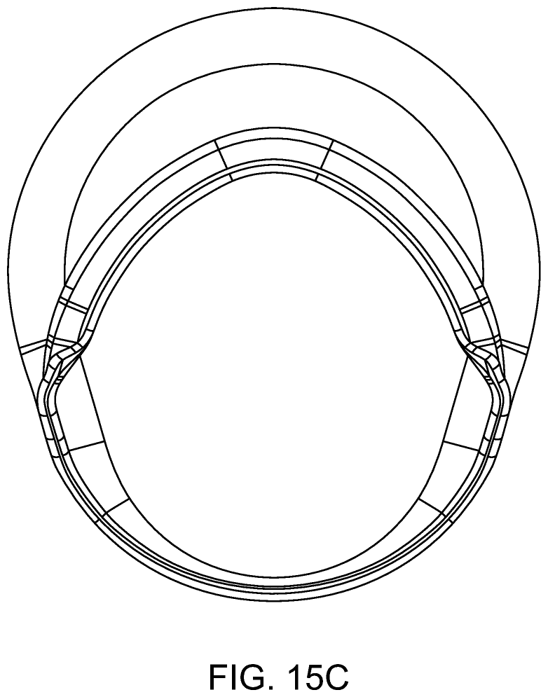

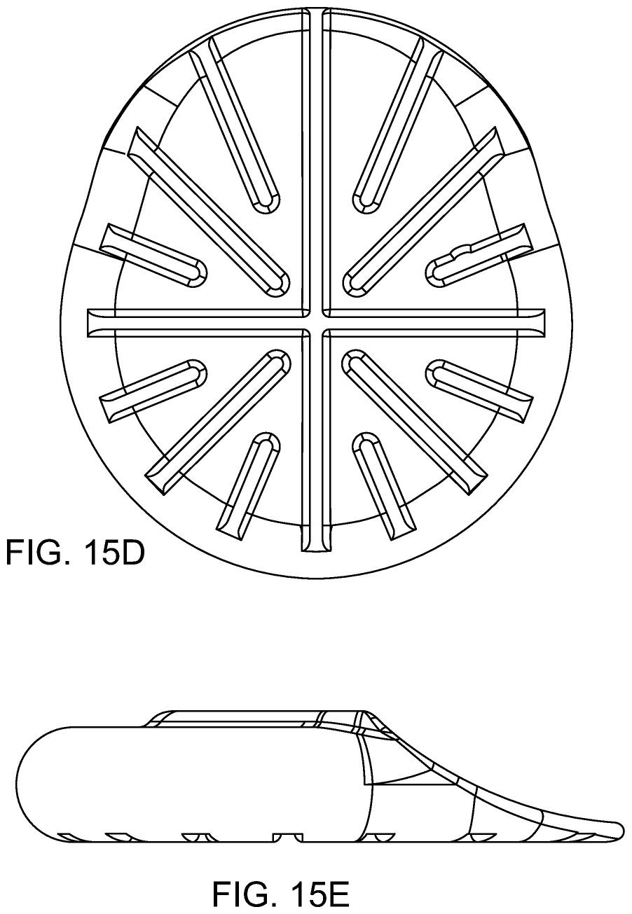

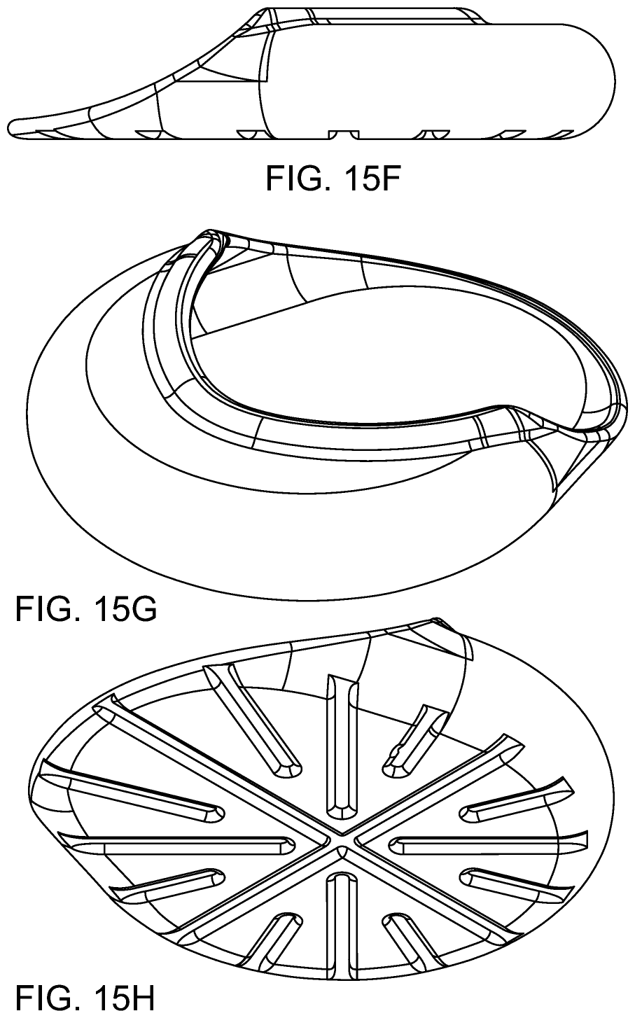

Such indentation configurations as shown in FIGS. 5A-6B can advantageously provide for venting of moisture as a subject wears the device, increased comfort for the patient, and protection for the wearable. While FIGS. 5A-6B illustrate covers with indentations, the covers may alternatively or in addition include protrusions to assist with venting, as shown in FIGS. 14 and 15. As illustrated in FIG. 14, a patient-facing surface of a wearable cover 1200 includes a plurality of semi-spherical protrusions 1210. In another example configuration, a patient-facing surface of a wearable cover 1300 includes a plurality of projections 1310 arranged in a generally radially-extending pattern. The projections can be arranged in an ordered pattern or can be randomly arranged. Because it can be desirable for neurological monitoring systems to be worn by a patient for extended periods of time (e.g., days, weeks), a cover can provide for protection, comfort, and easy cleaning of the device. A build-up of moisture between the wearable and the skin of a patient can affect attachment of the device to the patient and can be uncomfortable or harmful for the patient. A radial configuration of indentations and/or protrusions, such as extending from an interior location of the patient-facing surface toward a perimeter, can advantageously allow for those locations at which moisture is most likely to accumulate to be vented toward a perimeter of the cover. Alternatively, or in addition, the wearable device itself can include indentations and/or protrusions as illustrated with respect to the cover. Other example of covers are shown in FIGS. 14A-14H and FIGS. 15A-15H.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. An implant for a neural interface system, comprising:
a housing including a base and a flexible portion; and
two anchor attachment structures disposed at the base, the two anchor attachment structures arranged at an angle of greater than 90° and less than 180° with respect to one another as defined from an approximate center of the base, the two anchor attachment structures connected by a curved connecting member disposed at a patient-facing surface of the base.

2. The implant of claim 1, wherein the angle is within a range of about 130° to about 150°.

3. The implant of claim 2, wherein the angle is about 145°.

4. The implant of claim 1, wherein the at least two anchor attachment structures each comprise an attachment leg.

5. The implant of claim 4, wherein the attachment leg comprises a compliant material configured to conform to a curvature of a skull.

6. The implant of claim 4, wherein the anchor attachment leg comprises a material that resists oseointegration.

7. The implant of claim 6, wherein the material comprises polyetheretherketone (PEEK), polysulfone (PSU), polyethylenimine (PEI), thermoplastic polyurethane (TPU), or a combination thereof.

8. The implant of claim 4, wherein the attachment leg includes an inflection point.

9. The implant of claim 8, wherein the inflection point orients the attachment leg to deflect toward a patient-facing surface of the base.

10. The implant of claim 9, wherein the attachment leg is deflected toward the patient-facing surface of the base by about 5° to about 30°.

11. The implant of claim 10, wherein the attachment leg is deflected toward the patient-facing surface of the base by about 20°.

12. The implant of claim 8, wherein the attachment leg is flexible at the inflection point.

13. The implant of claim 1, wherein the flexible portion comprises at least one inflection point.

14. The implant of claim 13, wherein the inflection point orients the flexible portion to deflect toward a patient-facing surface of the base.

15. The implant of claim 14, wherein the flexible portion is deflected toward the patient-facing surface of the base by about 1° to about 90°, or by about 5° to about 45°, or by about 15°.

16. The implant of claim 1, wherein the flexible portion comprises a curved geometry.

17. The implant of claim 1, further comprising:
a lead connection area configured to engage leads extending from the implant; and
electronics disposed within the base of the housing and configured to record electroencephalography (EEG) signals detected from sensors of the leads.

18. The implant of claim 17, wherein the lead connection area comprises four lead connection structures arranged in a substantially flat configuration.

19. The implant of claim 17, wherein the lead connection area comprises four lead connection structures arranged in a bundled configuration.

20. The implant of claim 17, wherein the lead connection area comprises four lead connection structures arranged in a stacked configuration.

21. An implant for a neural interface system, comprising:
a housing including a base and a flexible portion; and
two anchor attachment structures disposed at the base, the two anchor attachment structures arranged at an angle of greater than 90° and less than 180° with respect to one another as defined from an approximate center of the base, each of the two anchor attachment structures including an attachment leg comprising an inflection point to deflect at least a portion of the anchor attachment structure toward a patient-facing surface of the base.

22. The implant of claim 21, wherein the inflection point of each of the two anchor attachment structures is disposed at an edge of the base.

23. A method of implanting a component of a neural interface system, comprising:
creating an incision in a scalp of a patient, the incision being of about 30 mm to about 50 mm in length; and
through the incision, placing an implant comprising a housing including a base and a flexible portion subcutaneously in a subscalp space against the skull of the patient.

24. The method of claim 23, wherein the incision is curved.

25. The method of claim 23, further comprising securing the implant to the skull of the patient at at least one attachment structure disposed at the base of the implant.

26. The method of claim 25, wherein the at least one attachment structure comprises two attachment structures arranged at an angle of greater than 90° and less than 180° with respect to one another.

27. A wearable headpiece of a neural interface system, comprising:
a housing; and
a removeable cover configured to enclose at least a patient-facing surface of the housing.

28. The wearable headpiece of claim 27, wherein the removeable cover comprises a plurality of indentations, a plurality of protrusions, or a combination thereof.

29. The wearable headpiece of claim 28, wherein the plurality of indentations, the plurality of protrusions, or the combination thereof forms paths extending from an interior location of a patient-facing surface of the cover toward a perimeter of the patient-facing surface of the cover.

30. The wearable headpiece of claim 29, wherein the paths extend radially from an interior location of the patient-facing surface of the cover.

31. The wearable headpiece of claim 29, wherein the plurality of indentations, the plurality of protrusions, or the combination thereof comprises a plurality of differently-sized indentations, a plurality of differently-sized protrusions, or a combination thereof.

32. The wearable headpiece of claim 31, wherein the plurality of differently-sized indentations or the plurality of differently-sized protrusions is arranged to increase in size from an interior location of a patient-facing surface of the cover toward a perimeter of the patient-facing surface of the cover.
